(19) United States
(12) Patent Application Publication
(10) Pub. No.: US 2006/0079803 A1
(43) Pub. Date: Apr. 13, 2006

(54) Method for improving the presentation of test stimuli during one-on-one clinical psychological tests

(76) Inventor: Amir Poreh, Solon, OH (US)

Correspondence Address: DR. MARK FRIEDMAN LTD., C/o Bill Polkinghorn, 9003 Florin Way, Upper Marlboro, MD, 20772, US

(21) Appl. No.: 11/225,162

(22) Filed: Sep. 14, 2005

Related U.S. Application Data
(60) Provisional application No. 60/614,297, filed on Sep. 30, 2004.

(51) Int. Cl. A61B 13/00

(52) U.S. Cl. 600/558

(57) ABSTRACT

A method for improving the administration of clinical psychological tests by storing multiple sets of visual stimuli and instructions in a computer that is operated and controlled by an examiner. The examiner selects desired stimuli or instructions from the sets and present them in a desired order, rate and format on a secondary display that is viewed by the examinee. The method eliminates the use of cumbersome stimuli booklets or a set of cards currently used by clinical test publishers. The method reduces the cost of developing new tests, modifying existing tests, and eliminates errors in stimuli presentation. The method enables control of the pace of stimuli presentation, and modification in real time, if necessary, of the course of tests according to the performance of the examinee and makes it possible for minimally trained technicians and psychologists to administer such tests at high standards.

### CROSS REFERENCE TO RELATED APPLICATIONS

This application claims the benefit from U.S. provisional application No. 60/614,297 filed 30-Sept.-2004 by the present inventor.

### FIELD AND BACKGROUND OF THE INVENTION

The present invention relates to the field of psychological evaluation and testing and more specifically to a method which improves the administering of “one-on-one”, ecologically valid, clinical psychological tests by using a primary and secondary display attached to a computer.

In the course of many traditional psychological evaluations, an examinee performs tasks in an ecologically valid or life-like manner. Ecologically valid testing is performed for instance, by using pencil and paper, responding verbally to questions, manually manipulating objects, or solving puzzles that require manual dexterity or grapho-motor speed. The scoring and interpretation of the examinees' performance is usually determined by comparing the results with normative data that specify the mean performance of large groups of people of similar backgrounds i.e. age, gender, education etc. It is therefore required that all psychological evaluations or tests be conducted in a standardized, and ecologically valid manner. For this reason, the “Standards for Educational and Psychological Testing” (Standard 15.1, p. 83) (Reference 2), specifies that “ . . . test examiners should follow carefully the standardized procedures for administration and scoring specified by the test publisher”.

Therefore, it is necessary to ensure that the presentation of test stimuli to an examinee should also be carried out in a standardized fashion. Varying the rate of stimuli presentation, for example, can alter the responses of the examinee, thereby tainting the reliability of the test.

In reality, however, stimuli are not always presented to the examinee in a standardized manner. Reference is now made to FIG. 1 (prior art) which illustrates a booklet 102 conventionally used for conveying visual stimuli by the examiner to the examinee at each stage of a test by showing the examinee a page in booklet 102, where the appropriate stimuli appear. It should be noted that such booklets often include more than forty pages. As a result, technicians and cursorily trained psychologists find it difficult to locate the appropriate test stimuli, and end up slowing down the pace of presenting stimuli and thus extending the time of tests. When such and similar problems occur, the test reliability deteriorates (references 1, 5, and 6). Similar and even more acute problems occur when stimuli of tests are presented on cards, as it is difficult to maintain correct order of the cards.

Correct administration of many “one on one” tests requires that the examiner has full control over the course of a test administration and can easily alter the test administration according to the performance and state of mind of the examinee. For example, if the examinee looks tired, inattentive, restless or exhibits psychomotor agitation, as a function of his/her neurological or psychological state, the examiner might have to change the course of the test in order to maintain its validity. Similarly, if an examinee does not respond correctly to a particular stimulus, the examiner might need to choose a different subtest with different stimuli. To accomplish such changes, the examiner has to quickly find the appropriate page in the original booklet 102 or even in a different booklet, where the appropriate set of stimuli is located. At the same time, he/she must also fully document the change in test procedure.

The complexity of carrying out and smoothly presenting correct stimuli is partially demonstrated by the instructions for administering the Block-Design Test (Reference 6). This test is usually administered using a set of plastic blocks, which are placed before the examinee on a table top together with booklet 102a turned to the section that shows a different configuration of blocks on each page. At each stage of the test, the examinee is asked to position the blocks on the table top according to the configuration that appears on a particular page of booklet 102. At the start of the test, the examiner decides, according to the background of the examinee, which configuration should be presented first. For instance, the examiner starts the test with configuration 11 that appears on page 17. The examiner flips the pages of the booklet 102, and places page 17 in front of the examinee. Following a demonstration by the examiner, using verbal instructions, the examiner asks the examinee to position the blocks on the table top according to the configuration shown on that page. Once the examinee completes the task within a specific time period, e.g. 60 seconds, the examiner records the response, typically using a pencil and paper. If the examinee fails on one of the first two configurations, e.g. configurations 11 or 12, the examiner must go to a configuration that appears on another page, for instance on page 8, and then sequentially present to the examinee a different series of configurations, e.g. configurations 4 and 5. Since there are a large number of stimuli (configurations) in this test, and many configurations, the examiner may have difficulty locating the appropriate stimuli, recording with a pencil and paper the performance of the examinee, and measuring with a stopwatch the time used by the examinee to assemble each configuration, while at the same time making sure that the time does not extend beyond the maximum time allowed for performing each stage (e.g. 60 to 120 sec).

Another example that demonstrates the inherent shortcomings of conventional prior art test methods is the test that assesses the abstract problem solving skills of clinical patients (Reference 5). Each page of large test booklet 102a, which is used in such a test, shows on the side presented to the examinee several items, and on the other side of that page instructions to the examiner. Following a question by the examiner, the examinee has to choose one of the items shown on that page and the examiner has to identify which item on the page was chosen. To do this, the examiner might have to move test booklet 102, or position him/herself near the examinee in order to observe the response. Then, the examiner has to use a complex set of decision rules to determine when to terminate and how to score the performance of the examinee on each section of the test (e.g. four sequentially incorrect items or three incorrect items within five responses). It should be recalled that the examiner must record the responses using, for instance, a pencil and paper while presenting the test stimuli in the conventional manner.

Clearly, the complexity of carrying out such tests demands considerable training and experience from the examiner. For this reason, professional psychologists go through lengthy technical training in their graduate and post graduate studies (Reference 1). Moreover, such abilities are affected by practice, so that the skill of psychologists that do not provide testing services regularly is likely to deteriorate, and retraining may be required.

For the above mentioned reasons, the state licensing boards of New York, Oregon, North Carolina and Arkansas have recently prohibited by regulations the use of technicians for administering psychological tests (Reference 3). Clearly, these regulations have increased the cost of administering psychological tests and, in practice; many complicated psychological tests are not administered at all.

Various methods have been proposed for computer-based methods for securing high administration standards of psychological tests. See for example, U.S. Pat. No. 5,961,332, U.S. Pat. No. 5,991,565 and U.S. Pat. No. 6,115,68 and U.S. Pat. No. 6,629,846. These methods relate solely to the recording, scoring and/or evaluating of tests that are administered to the examinee by the computer. The prior art methods do not provide, however, a method which presents visual test stimuli to the examinee by an examiner, in which the examiner controls and is able to modify the rate and sequence of their presentation. Moreover, prior art methods do not enable the presentation of a series of stimuli which are determined in real time according to the performance of the examinee, while maintaining the ecological validity of the testing and ensuring real time accurate recording of the testing procedure, results and scoring.

Other methods have been suggested whereby an examinee is provided instructions and performs the test using a graphic interface (e.g. monitor) of a computer-based system with no interference of the examiner. Representative examples of these methods include: U.S. Pat. No. 5,211,564, U.S. Pat. No. 5,218,535, U.S. Pat. No. 5,326,270, U.S. Pat. No. 5,379,213, U.S. Pat. No. 5,565,316 and U.S. Pat. No. 6,030,226. Although such methods might have considerable merit for testing the general population, they are not adequate for psychological testing of cognitively impaired examinees, computer illiterate or technologically illiterate populations. Indeed, it has been shown (Reference 4) that many tasks performed on a graphic interface of a computer-based system, e.g. memory tasks that require the recollection of words, stories; puzzles; manual dexterity tasks, lack ecological validity and do not reproduce the results obtained when these same tasks are performed in a conventional, life like manner.

Reference is now made to FIG. 2, a prior art drawing showing a prior art system for administering ecologically valid psychological examinations. In U.S. Pat. publication No. 6,629,846 of Michael Poreh, a method is disclosed for recording the process by which an examinee 101 performs a psychological test that includes a predetermined finite number of elements that are to be sequentially manipulated. Such tests include memory tests or graphic manipulation tests, such as copying complex figures. An examiner 103 observing the examinee uses an input device 107, such as a touch-sensitive screen or a mouse, to register and store the time at which each element is manipulated. In such a way, the process is accurately recorded in an ecologically valid manner.

Amir Poreh in U.S. patent application Ser. No. 10/859,215 filed on Jun. 3, 2004 disclosed a method of administering ecologically valid psychological examinations to examinee 101 by examiner 103 equipped with a computer 106 when examinee 101 neither interacts with computer 106 nor sees the display of the examiner 104. Stimuli/instructions are visually presented to the examiner on display 104 connected to computer 106. Examiner 103 performs an action: either reading out loud the stimuli/instructions, or presses input device 107 connected to computer 106 which audibly plays the stimuli/instructions to examinee 101 over a sound system connected to computer 106.

These method, although useful, do not fully mitigate the difficulties encountered in carrying out tests in which series of stimuli/instructions have to be presented visually to the examinee during the course of these tests, as described earlier.

Thus, there is a need for a reliable and efficient method for presenting visual stimuli, in a standard, precise and reliable manner, which is fully controlled by the examiner and which enables convenient, real time recording of the order of presentation and of the response of the examinee to each stimulus for scoring purposes, so as to maintain the ecological validity of psychological such tests and enhance the reliability of their administration and scoring. Moreover, there is a need for a method that will make it possible for minimally trained technicians to adequately administer such psychological tests. There is also a need for a method that will enable the examiner to change individual test items without issuing new booklets.

The present invention uses a secondary display connected to a computer in addition to a primary display. The following U.S. patent publications, which disclose the use of secondary displays, are included herein by reference as if entirely set forth herein: U.S. Pat. No. 6,522,309, U.S. Pat. No. 5,673,170 and U.S. Pat. No. 4,884,068.

### REFERENCES

1. Gary Groth-Marnat (1990). Handbook of Psychological Assessment. John Wiley & Sons, Inc.
2. Standards for Educational and Psychological Testing: American Educational Research Association, Released: February, 2000.
3. Jennifer Daw Holloway (2003) Psychologists are making headway in getting the prohibition on the use of nondoctoral testing technicians reversed in some states. APA Monitor, Volume 34, No. 1, p. 26.
4. Sbordone, R. J. and Long, C. J. Ecological Validity of Neuropsychological Testing (1996) Delray Beach, Fla.: GR Press/St. Lucie Press.
5. Spreen O. & Strauss E., (1998). A Compendium of Neuropsychological Tests. Oxford University Press, New York.
6. Moon, G. W., Blakey, W. A., Gorsuch, R. L., & Fantuzzo, J. W. (1991). Frequent WAIS-R administration errors. Professional Psychology Research and Practice, 22, 256-258.

The term “computer” as used herein refers to a programmable electronic machine that performs, assembles, stores, correlates, displays and/or processes information. A “computer” as used herein includes a “desktop” computer, a portable (laptop) computer, a notebook computer, personal digital assistant or handheld computer. The term “input device” refers to any device typically used to input commands or information to a computer, such as a keyboard, mouse, and/or touch screen. The term “stimulus” is used herein to include “instruction” although the term “stimulus” is generally used in order to receive and record a response whereas the term “instruction” is generally used when recording a response is not necessarily required. The terms “display”, “screen” and “monitor” are used interchangeably to describe a physical visual human interface attached to a computer.

The terms “test”, “evaluation” and “examination” are used herein interchangeably.

### SUMMARY OF THE INVENTION

According to the present invention there is provided, a method for administering ecologically valid psychological examinations to an examinee by an examiner equipped with a computer. The computer is attached to input devices, a primary display, viewed by the examiner, and a secondary display, viewed by the examinee. The method includes visually presenting at each stage of a test a stimulus and/or instructions to the examinee on the secondary display. The visual presentation is performed by the computer upon command of the examiner, and the examiner is visually presented at least a portion of the stimulus and/or instructions on the primary display, or symbols and text related to the stimulus/instructions, preferably without interfering with other applications running on the computer and without obscuring sections (e.g. windows) of the main display. Preferably, the examinee responds to the stimulus or instruction and the computer times the duration of the examinee's response to the stimulus and/or instructions. Upon the duration being greater than a previously determined value, the examiner records this fact and continues to an appropriate stage by selecting a second stimulus/instruction and triggers the computer to visually present the second stimulus/instructions to the examinee on the secondary display. Upon the duration to be less than a previously determined value, and the examiner observes that the examinee has completed the response, the examiner records the response and commands the computer to continue the test in its usual course, namely present the next stimulus/instructions, or selects a a different instruction/stimulus and inputs using the input device and thereby triggers the computer to visually present on the secondary display the second instruction/stimulus to the examinee.

display in view of the examiner, a secondary display in view of the examinee, and at least one display driver which controls the primary display and the secondary display. The stimulus/instruction is selected by the examiner and visibly presented to the examinee on the secondary display. Preferably, a timer and an input device are integrated with the computer. The computer times the response of the examinee to the stimulus/instruction; and the examiner inputs to the computer using the input device to indicate that the examinee has completed a response to the stimulus/instruction.

ecologically valid psychological examinations to an examinee by an examiner equipped with a computer. The computer is attached to an input device, a primary display, and a secondary display, and the method includes visually presenting a stimulus or an instruction to the examinee on the secondary display.

### BRIEF DESCRIPTION OF THE DRAWINGS

The invention is herein described, by way of example only, with reference to the accompanying drawings, wherein:

FIG. 1 is a drawing of a prior art conventional system for administering psychological examinations;

FIG. 2 is drawing of a second prior art conventional system for administering psychological examinations;

FIG. 3 is a drawing of a system, according to an embodiment of the present invention for administering ecologically valid psychological examinations; and

FIG. 4 is a flow drawing that describes schematically some features of a method for administering psychological examinations according to an embodiment of the present invention.

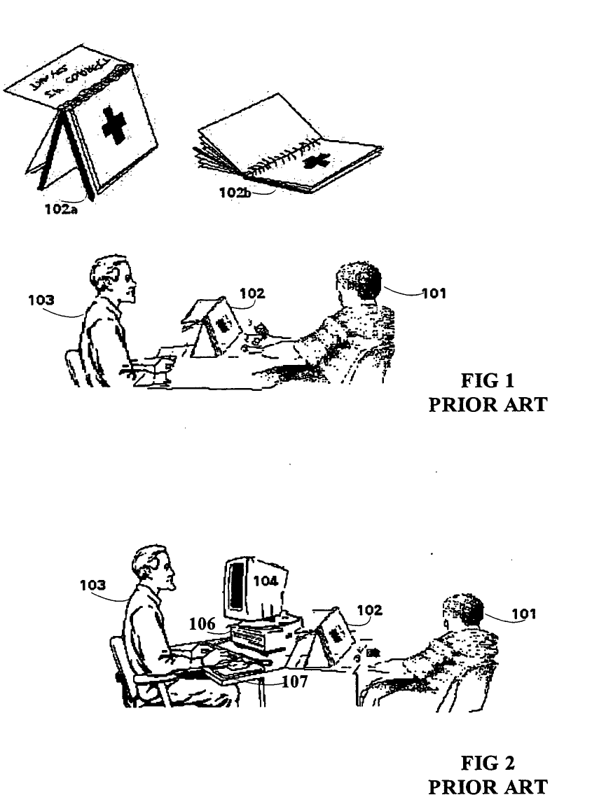

### DESCRIPTION OF THE PREFERRED EMBODIMENTS

The present invention relates to the field of psychological evaluations and testing and more specifically to a method which improves the administering of “one-on-one”, ecologically valid, clinical psychological tests by storing multiple sets of visual stimuli in a computer that is operated and controlled by an examiner so that the examiner can select desired stimuli from the sets and present them in a desired order, rate and format on a secondary display that is viewed by the examinee. At each stage of the test, a stimulus and/or an instruction may be presented to the examinee on the secondary display and at simultaneously shown to the examiner on the primary display without interfering with the display of other applications running on the computer being used for instance to record and score the response of the examinee and for managing the administration of the test. Alternatively, text or graphics related to the stimulus/instruction is presented simultaneously to the examiner. For example the word “Dog” appears on the primary display instead of a picture of a dog that is presented as a stimulus to the examinee on the secondary display.

Although the discussion herein relates primarily to a single computer connected to a primary and secondary display, the present invention is equivalently performed with two computers operatively attached each with a display.

Before explaining as examples embodiments of the invention in detail, it is to be understood that the invention is not limited in its application to the details of design and the arrangement of the components set forth in the following description or illustrated in the drawings. The invention may be executed using other embodiments or being practiced or carried out in various ways. Also, it is to be understood that the phraseology and terminology employed herein is for the purpose of description and should not be regarded as limiting.

An embodiment of the present invention is preferably used when an examinee performs a psychological test in an ecologically valid manner, such as by using the methods of U.S. Pat. No. 6,629,846 and/or U.S. patent application Ser. No. 10/859,215. U.S. Pat. No. 6,629,846 and U.S. patent application Ser. No. 10/859,215 are included herein by reference for all purposes as if entirely set forth herein.

The principles and operation of a system and method of clinical psychological testing, according to the present invention, may be better understood with reference to the drawings and the accompanying description.

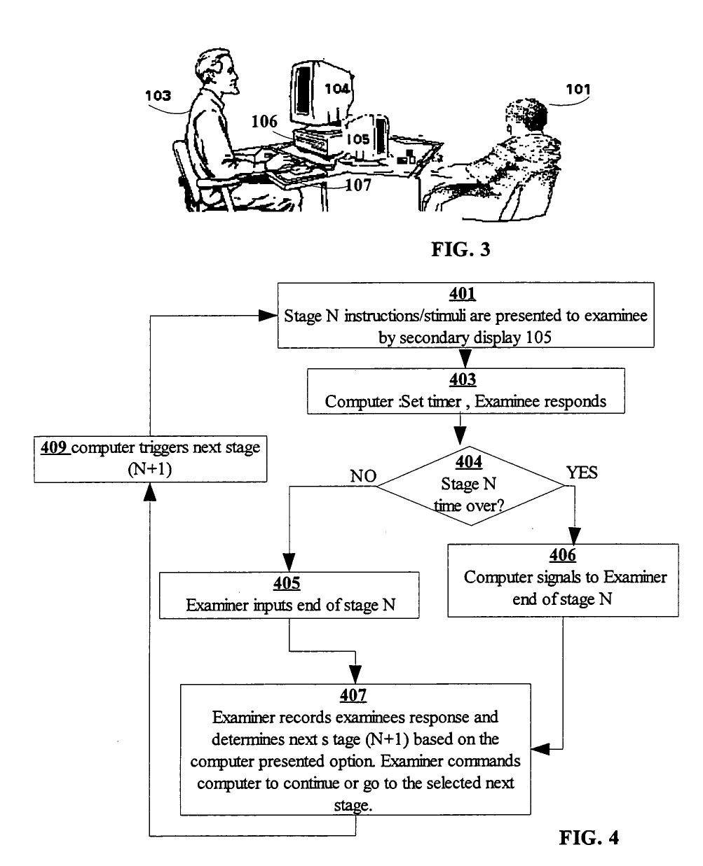

Reference is now made to FIG. 3, a schematic drawing of a system and FIG. 4 a flow drawing of a method, both according to embodiments of the present invention.

According to the present invention, the instructions and/or stimuli have been previously stored in storage (not shown) of computer 106. The instructions/stimuli are presented (step 401) at the command of the examiner at the right moment on a secondary display 105, which is connected and controlled by the examiner's computer 106, so that the instructions/stimuli are viewed by examinee 101. At step 401 when instructions/stimuli are presented on secondary display 105, the instruction/stimuli are also presented visually on primary computer display 104 viewed solely by the examiner without interfering with other computer applications, such as those used for the administration, recording and scoring of the test, e.g. by obscuring their displays on the primary screens. Computer 106 typically sets a timer which measures time from step 401 during which examinee 101 is supposed to respond by performing the instruction. Upon completing a stage of the test, as observed by examiner 103, he will typically provide an input (step 405) using input device 107, or when the time allocated for completing the stage lapses (decision block 404), computer 106 signals (step 406) examiner 103 that time has lapsed and examiner 103 records the performance on stage N, and determines the next stage. New visual stimuli/instructions for the next stage of the examination and appropriate data are retrieved (step 407) from storage attached to computer 106. Upon command from examiner 103, computer 106 initiates (step 409) the next stage of the examination. When necessary, examiner 103 can modify the course of the test and the presentation of the stimuli using the application running on computer 106. In this fashion, the test will always be administered at the highest standards. Effects of the fatigue on the part of examiner 103, insufficient training and/or errors in reading the instructions are substantially eliminated and individuals with minimal training could also administer standard, ecological tests with high quality.

According to an embodiment of the present invention, multiple sets of visual stimuli are stored in computer 106 that is operated and controlled by examiner 103. Computer 106 may equivalently be a personal computer, personal digital assistant, hand held computer, a computer tablet or any computerized device, so that examiner 103 can select desired stimuli from the available sets of stimuli/instructions and present them at a desired rate and format on secondary display 105 that is viewed by examinee 101.

The present invention, in preferred embodiments, uses secondary display 105 which operates independently of primary display 104. Both displays are preferably controlled by the same application, specifically suitable for performing the psychological examination. However, whereas secondary display 105 is programmed to display solely instructions and stimuli presented to examinee 101, primary display 104 displays all the features of the testing application, such as interfaces for recording and scoring logging responses of examinee 101, by the examiner, and information for determining the next stage of the psychological examination.

Configuring a secondary display 105 to display content different from primary display 104, is performed for instance with independent display cards and respective drivers which are both accessible to the psychological testing application so that the secondary displays shows only the stimuli or instructions which the examinee has to see, whereas the primary display interfaces to other applications that the examiner might use for administering, recording and scoring in addition to these stimuli or instructions.

Clearly, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents falling within the scope of the invention may be resorted to.

While the invention has been described with respect to a limited number of embodiments, it will be appreciated that many variations, modifications and other applications of the invention may be made.

What is claimed is:

1. A method for administering ecologically valid psychological examinations to an examinee by an examiner equipped with a computer, the computer operatively attached to an input device and a primary display, the method comprising the steps of:
(a) providing a secondary display operatively attached to the computer; and
(b) visually presenting at least one stimulus to the examinee on said secondary display.

2. The method, according to claim 1, wherein said visual presenting is performed by the computer upon command of the examiner.

3. The method, according to claim 1, further comprising the step of:
(c) visually presenting to the examiner on the primary display at least one item associated with said at least one stimulus.

4. The method, according to claim 1, further comprising the step of:
(c) simultaneously visually presenting to the examiner at least a portion of said at least one stimulus.

5. The method, according to claim 4, wherein said simultaneously visually presenting to the examiner is performed without interfering with other applications running on the computer.

6. The method, according to claim 1, further comprising the steps of:
(c) responding by the examinee to said at least one stimulus; and
(d) timing by the computer a duration of said responding.

7. The method, according to claim 6, further comprising the step of:
(e) upon said duration being greater than a previously determined value, selecting by the examiner a second at least one stimulus and triggering by an examiner said second at least one stimulus to be visually presented to the examinee on said secondary display.

8. The method, according to claim 6, further comprising the steps of:
(e) upon said duration being less than a previously determined value, observing by the examiner that the examinee has completed said responding; and
(f) selecting by the examiner a second at least one stimulus by inputting using the input device, thereby triggering the computer to visually present on said secondary display said second at least one stimulus to the examinee.

9. A system for administering ecologically valid psychological examinations to an examinee by an examiner, the system comprising:
(a) a computer operatively attached to a primary display in view of the examiner and a secondary display in view of the examinee; and
(b) at least one display driver which controls the primary display and the secondary display; wherein at least one stimulus is selected by the examiner and visibly presented to the examinee using said secondary display.

10. The system, according to claim 9, further comprising:
(c) a timer integrated with said computer which times the response of the examinee to said stimulus; and
(d) an input device, operatively attached to the computer, wherein the examiner inputs to said computer using said input device to indicate that the examinee has completed a response to said at least one stimulus.

11. A program storage device readable by a machine, tangibly embodying a program of instructions executable by the machine to perform a method for administering ecologically valid psychological examinations to an examinee by an examiner equipped with a computer, the computer operatively attached to an input device and a primary display, and a secondary display, the method comprising visually presenting a stimulus to the examinee on said secondary display.
